(19) United States
(12) Patent Application Publication
(10) Pub. No.: US 2024/0024363 A1
(43) Pub. Date: Jan. 25, 2024

(54) TREATMENT OF LIMB ISCHEMIA BY BONE MARROW STEM CELLS AND MODIFICATION OF DISEASED MICROENVIRONMENT

(71) Applicant: CREATIVE MEDICAL TECHNOLOGIES, INC., Phoenix, AZ (US)

(72) Inventors: Thomas ICHIM, San Diego, CA (US); Amit PATEL, Salt Lake City, UT (US); Courtney BARTLETT, Niceville, FL (US)

(73) Assignee: CREATIVE MEDICAL TECHNOLOGIES, INC., Phoenix, AZ (US)

(21) Appl. No.: 18/348,292

(22) Filed: Jul. 6, 2023

Related U.S. Application Data
(60) Provisional application No. 63/390,759, filed on Jul. 20, 2022.

(51) Int. Cl. A61K 35/28; A61K 45/06; C12N 5/078

(52) U.S. Cl. CPC A61K 35/28; A61K 45/06; C12N 5/0634

(57) ABSTRACT

Methods and compositions are provided to treat limb ischemia such as Beurger's Disease through the use of bone marrow stem cells that are administered prior to, concurrently with, and subsequently to administration of agents that modify the microenvironment of the ischemic limb(s). In one embodiment, said agents are antioxidants, more specifically antioxidants capable of diffusing across ischemic areas and preserving regenerative activity of administered stem cells. In another embodiment, the invention provides hormonal modifications to overcome deficiencies associated with ischemia predisposing pathologies.

### CROSS-REFERENCE TO RELATED APPLICATIONS

This application is a non-provisional of and claims priority to U.S. Provisional Application Ser. No. 63/390,759, titled “Treatment of Limb Ischemia by Bone Marrow Stem Cells and Modification of Diseased Microenvironment” filed Jul. 20, 2022, which is hereby incorporated by reference in its entirety.

### FIELD OF THE INVENTION

The invention belongs to the field of treating ischemia, through the use of immune enhanced bone marrow stem cells.

### BACKGROUND OF THE INVENTION

Ischemic diseases as well as other diseases involving a cardiovascular and, more specifically, arterial insufficiency, affect a rising patient population. One important reason for a progression of ischemic disease is the partial or complete occlusion of arterial vessels resulting in a reduced supply of oxygen and nutrients of the tissue supplied by the arterial vessel. Angina pectoris, chest pain, is a clinical syndrome reflecting inadequate oxygen supply for myocardial metabolic demands with resultant ischemia and is generally caused by obstruction (stenosis), spasm of coronary arteries, and endothelial or microvascular dysfunction. Angiogenesis is a process in which already pre-existing small arteriolar collaterals can develop to full functional conductance arteries which bypass the site of an arterial occlusion and/or compensate blood flow to ischemic territories supplied by the insufficient artery. Consequently, angiogenesis is a highly effective endogenous mechanism for the maintenance and regeneration of the blood flow after an acute or chronic occlusive event in an arterial vessel. In this case the collaterals can function as natural bypasses.

Utilization of autologous bone marrow to stimulate angiogenesis has been previously described, however, this approach is limited by reduced efficacy with aging and/or comorbidities. There is a need for providing agents that enhance ability of bone marrow cells to promoting collateral circulation.

### SUMMARY

Preferred embodiments are drawn to methods of treating an ischemic disease comprising of administration of bone marrow mononuclear cells/aspirate in a microenvironment that has been altered in order to augment the efficacy of said bone marrow mononuclear cells/aspirate to induce a therapeutic benefit in said ischemic disease.

Preferred methods include embodiments wherein said ischemic disease is heart failure.

Preferred methods include embodiments wherein said ischemic disease is stroke.

Preferred methods include embodiments wherein said ischemic disease is limb ischemia.

Preferred methods include embodiments wherein said ischemic disease is buerger's disease.

Preferred methods include embodiments wherein said antioxidant is super oxide dismutase.

Preferred methods include embodiments wherein said super oxide dismutase is manganese dependent.

Preferred methods include embodiments wherein the gene encoding manganese dependent superoxide dismutase is administered intra-muscularly.

Preferred methods include embodiments wherein said gene encoding manganese dependent superoxide dismutase is transfected into tissue by means of a viral vector.

Preferred methods include embodiments wherein said gene encoding manganese dependent superoxide dismutase is transfected into tissue by means of a hydrodynamic transfection.

Preferred methods include embodiments wherein said gene encoding manganese dependent superoxide dismutase is transfected into tissue by means of a mRNA liposomal delivery.

Preferred methods include embodiments wherein said gene encoding manganese dependent superoxide dismutase is transfected into tissue by means of naked DNA administration.

Preferred methods include embodiments wherein said antioxidant is zinc.

Preferred methods include embodiments wherein said antioxidant is vitamin C.

Preferred methods include embodiments wherein said antioxidant is vitamin E.

Preferred methods include embodiments wherein said antioxidant is selenium.

Preferred methods include embodiments wherein said antioxidant is Trolox.

Preferred methods include embodiments wherein said antioxidant is ebselen.

Preferred methods include embodiments wherein said antioxidant is glutathione.

Preferred methods include embodiments wherein said antioxidant is carotene.

Preferred methods include embodiments wherein said antioxidant is ubiquinol.

Preferred methods include embodiments wherein said antioxidant is propyl gallate.

Preferred methods include embodiments wherein said antioxidant is hydrogen gas.

Preferred methods include embodiments wherein said antioxidant is xenon gas.

Preferred methods include embodiments wherein said antioxidant is argon gas.

Preferred methods include embodiments wherein said antioxidant is neon gas.

Preferred methods include embodiments wherein said antioxidant is krypton gas.

Preferred methods include embodiments wherein said antioxidant is butylated hydroxytoluene.

Preferred methods include embodiments wherein said antioxidant is butylated hydroxyanisole.

Preferred methods include embodiments wherein said antioxidant is butylated hydrogen sulfide.

Preferred methods include embodiments wherein said antioxidant is erythorbate.

Preferred methods include embodiments wherein said antioxidant is sodium tripolyphosphate.

Preferred methods include embodiments wherein said antioxidant is ethylenediaminetetraacetic acid.

Preferred methods include embodiments wherein said antioxidant is ethoxyquin.

Preferred methods include embodiments wherein said antioxidant is casein.

Preferred methods include embodiments wherein said antioxidant is pyruvate.

Preferred methods include embodiments wherein said antioxidant is minocycline.

Preferred methods include embodiments wherein said antioxidant is tetracycline.

Preferred methods include embodiments wherein said anti-inflammatory agent is hydroxychloroquine.

Preferred methods include embodiments wherein said anti-inflammatory agent is an NF-kappa B inhibitor.

Preferred methods include embodiments wherein said NF-kappa B inhibitor is hydroxychloroquine.

Preferred methods include embodiments wherein said NF-kappa B inhibitor is Calagualine.

Preferred methods include embodiments wherein said NF-kappa B inhibitor is Conophylline.

Preferred methods include embodiments wherein said NF-kappa B inhibitor is Evodiamine.

Preferred methods include embodiments wherein said NF-kappa B inhibitor is Geldanamycin.

Preferred methods include embodiments wherein said NF-kappa B inhibitor is selected from a group comprising of: Perrilyl alcohol, Protein-bound polysaccharide from basidiomycetes, Roc aglamides (Aglaia derivatives), 15-deoxy-prostaglandin J(2), Lead, Anandamide, Artemisia vestita, Cobrotoxin, Dehydroascorbic acid (Vitamin C), Herbimycin A, Isorhapontigenin, Manumycin A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2Cl), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin A1, 20(S)-Protopanaxatriol (ginsenoside metabolite), Rengyolone, Rottlerin, Saikosaponin-d, Saline (low Na+ isotonic).

Preferred methods include embodiments wherein an inhibitor of inflammatory cytokines is administered prior to, concurrent with, or subsequent to stem cell administration.

Preferred methods include embodiments wherein said inflammatory cytokines are selected from a group comprised of: inflammatory cytokines are cytokines capable of inducing expression of genes in endothelial cells selected from a group comprising of: IL-6, Myosin 1, IL-33, Hypoxia Inducible Factor-1, Guanylate Binding Protein Isoform I, Aminolevulinate delta synthase 2, AMP deaminase, IL-17, DNAJ-like 2 protein, Cathepsin L, Transcription factor-20, M31724, pyenylalkylamine binding protein; HEC, GA17, arylsulfatase D gene, arylaulfatase E gene, cyclin protein gene, pro-platelet basic protein gene, PDGFRA, human STS WI-12000, mannosidase, beta A, lysosomal MANBA gene, UBE2D3 gene, Human DNA for Ig gamma heavy-chain, STRL22, BHMT, Homo sapiens Down syndrome critical region, FI5613 containing ZNF gene family member, IL8, ELFR, Homo sapiens mRNA for dual specificity phosphatase MKP-5, Homo sapiens regulator of G protein signaling 10 mRNA complete, Homo sapiens Wnt-13 Mma, Homo sapiens N-terminal acetyltransferase complex ard1 subunit, ribosomal protein L15 mRNA, PCNA mRNA, ATRM gene exon 21, HR gene for hairless protein exon 2, N-terminal acetyltransferase complex and 1 subunit, HSM801431 Homo sapiens mRNA, CDNA DKFZp434N2072,RPL26, and HR gene for hairless protein, regulator of G protein signaling.

Preferred methods include embodiments wherein an immune suppressive agent is administered prior to, concurrent with or subsequent to stem cell administration.

Preferred methods include embodiments wherein said immune suppressive agent is cyclophosphamide.

Preferred methods include embodiments wherein said immune suppressive agent is prednisone.

Preferred methods include embodiments wherein said immune suppressive agent is budesonide.

Preferred methods include embodiments wherein said immune suppressive agent is prednisolone.

Preferred methods include embodiments wherein said immune suppressive agent is tofacitinib.

Preferred methods include embodiments wherein said immune suppressive agent is cyclosporine.

Preferred methods include embodiments wherein said immune suppressive agent is tacrolimus.

Preferred methods include embodiments wherein said immune suppressive agent is everolimus.

Preferred methods include embodiments wherein said immune suppressive agent is azathioprine.

Preferred methods include embodiments wherein said immune suppressive agent is leflunomide.

Preferred methods include embodiments wherein said immune suppressive agent is Mycophenolate.

Preferred methods include embodiments wherein said immune suppressive agent is adalimumab.

Preferred methods include embodiments wherein said immune suppressive agent is anakinra.

Preferred methods include embodiments wherein said immune suppressive agent is certolizumab.

Preferred methods include embodiments wherein said immune suppressive agent is etanercept.

Preferred methods include embodiments wherein said immune suppressive agent is golimumab.

Preferred methods include embodiments wherein said immune suppressive agent is infliximab.

Preferred methods include embodiments wherein said immune suppressive agent is ixekizumab.

Preferred methods include embodiments wherein said immune suppressive agent is natalizumab.

Preferred methods include embodiments wherein said immune suppressive agent is rituximab.

Preferred methods include embodiments wherein said immune suppressive agent is secukinumab.

Preferred methods include embodiments wherein said immune suppressive agent is tocilizumab.

Preferred methods include embodiments wherein said immune suppressive agent is ustekinumab.

Preferred methods include embodiments wherein said immune suppressive agent is vedolizumab.

Preferred methods include embodiments wherein said immune suppressive agent is basiliximab.

Preferred methods include embodiments wherein said immune suppressive agent is daclizumab.

Preferred methods include embodiments wherein said ischemic disease is thromboangiitis obliterans.

Preferred methods include embodiments wherein said ischemic disease is lumbar ischemia.

Preferred methods include embodiments wherein said bone marrow mononuclear cells/aspirate is a bone marrow concentrate.

Preferred methods include embodiments wherein said microenvironment is associated with degranulation of neutrophils.

Preferred methods include embodiments wherein said degranulation of neutrophils is associated with increase oxidative stress.

Preferred methods include embodiments wherein said degranulation of neutrophils is associated with increased release of elastase.

Preferred methods include embodiments wherein said degranulation of neutrophils is associated with increased release of myeloperoxidase.

Preferred methods include embodiments wherein said degranulation of neutrophils is associated with increased release of TNF-alpha.

Preferred methods include embodiments wherein said degranulation of neutrophils is associated with increased release of IL-1 beta.

Preferred methods include embodiments wherein said degranulation of neutrophils is associated with increased release of IL-6.

Preferred methods include embodiments wherein said degranulation of neutrophils is associated with increased release of IL-8

Preferred methods include embodiments wherein said degranulation of neutrophils is associated with increased release of IL-9.

Preferred methods include embodiments wherein said degranulation of neutrophils is associated with increased release of IL-11.

Preferred methods include embodiments wherein said degranulation of neutrophils is associated with increased release of IL-17.

Preferred methods include embodiments wherein said degranulation of neutrophils is associated with increased release of IL-18.

Preferred methods include embodiments wherein said degranulation of neutrophils is associated with increased release of interferon gamma.

Preferred methods include embodiments wherein said degranulation of neutrophils is associated with increased release of CCL2.

Preferred methods include embodiments wherein said degranulation of neutrophils is associated with increased release of CCL3.

Preferred methods include embodiments wherein said degranulation of neutrophils is associated with increased release of CCL17.

Preferred methods include embodiments wherein said degranulation of neutrophils is associated with increased release of TSLP-1.

Preferred methods include embodiments wherein said degranulation of neutrophils is associated with increased release of CXCL1.

Preferred methods include embodiments wherein said degranulation of neutrophils is associated with increased release of CXCL3.

Preferred methods include embodiments wherein said degranulation of neutrophils is associated with increased release of MMP1.

Preferred methods include embodiments wherein said degranulation of neutrophils is associated with increased release of MMP-3.

Preferred methods include embodiments wherein said degranulation of neutrophils is associated with increased release of MMP-9.

Preferred methods include embodiments wherein said degranulation of neutrophils is associated with increased release of neutrophil extracellular traps.

Preferred methods include embodiments wherein said microenvironment is associated with activation of complement.

Preferred methods include embodiments wherein said microenvironment is altered by administration of a complement inhibitor prior to, concurrent with, and subsequent to administration of said bone marrow cells.

Preferred methods include embodiments wherein said complement inhibitor is cobra venom factor.

Preferred methods include embodiments wherein said complement inhibitor is serpin.

Preferred methods include embodiments wherein said complement inhibitor is Factor I.

Preferred methods include embodiments wherein said complement inhibitor is CD55.

Preferred methods include embodiments wherein said complement inhibitor is CD35.

Preferred methods include embodiments wherein said complement inhibitor is CD46.

Preferred methods include embodiments wherein said complement inhibitor is Factor H.

Preferred methods include embodiments wherein said complement inhibitor is an anti-C5 antibody.

Preferred methods include embodiments wherein said complement inhibitor is an anti-C5 Eculizumab.

### DETAILED DESCRIPTION OF THE INVENTION

The invention provides administration of augmenting agents together with autologous bone marrow mononuclear cells/aspirate as a means of enhancing regenerative activity of said bone marrow mononuclear cells/aspirate. In one embodiment, the invention provides the systemic and/or oral administration of augmenting agents to increase efficacy of injection of bone marrow mononuclear cells/aspirate for stimulation of angiogenesis/muscle regeneration in patients with peripheral artery disease/limb ischemia.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0” should be interpreted to include not only the explicitly recited values of about 0.01 to about 2.0, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. This same principle applies to ranges reciting only one numerical value. Furthermore, such an interpretation should apply regardless of the breadth of the range or the characteristics being described.

Before the present invention is described in detail, it is to be understood that this invention is not limited to the particular methodology, protocols, cell lines, vectors, and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

The term “treatment” or “prevention” means that not only symptoms of the disease are relieved but that also the disease itself is treated or prevented. In a preferred embodiment, the term “treatment” means improving the prognosis of said disease.

The term “arterial insufficiency” refers to any insufficient blood or oxygen supply or any other insufficient supply of a tissue which is provided by an artery. This insufficient supply can be overcome by the methods and uses of the present invention wherein n-acetylcysteine together with autologous bone marrow mononuclear cells/aspirate are used to increase the supply of a given tissue. The arterial insufficiency may occur both during physical rest and during an exercise. Furthermore, the arterial insufficiency may be due to an increased demand of oxygen or blood flow of a tissue supplied by the artery or a bypass or shunt. This increased demand of oxygen or blood flow can have several reasons including but not limited to increased sport or physical activity, and increased mental activity or a disease requiring an increased demand of oxygen or blood flow. Furthermore, the arterial insufficiency may be characterized by a partial (stenosis) or complete occlusion of an arterial vessel. In the context of the present invention, the term “partial occlusion” is equivalent to a stenosis.

The term “ex vivo” refers generally to activities or procedures that involve living cells or tissues taken from an organism and cultured in a laboratory apparatus, usually under sterile conditions, and typically for a few minutes or up to about 96 hours, but including up to 48 or 72 hours, depending on the circumstances. Tissue culture experiments or procedures lasting longer than a few days using living cells or tissue are typically considered to be “in vitro,” though in certain embodiments, this term can be used interchangeably with ex vivo.

The term “physical exercise” means any training of the subject, including but not limited to training in exercise rooms, jogging, walking, nordic walking, swimming, dancing, cycling and hiking. The skilled person will appreciate that any exercise will be helpful in the context of the invention, provided that it is performed in conjunction with the administration of NO in the donor. Preferably, the term “physical exercise” does not include unsupervised, unprescribed routine movements like casual walking or house work.

The term “Vascularization” refers to the process of generating new blood vessels in a tissue through neovascularization or angiogenesis. “Neovascularization” refers to the de novo formation of functional microvascular networks in tissues to restore perfusion to a tissue. Neovascularization differs from angiogenesis. “Angiogenesis” is mainly characterized by the protrusion and outgrowth of capillary buds and sprouts from pre-existing blood vessels.

Without being limited to any one or more explanatory mechanisms for the immunomodulatory, regenerative and other properties, activities, and effects of bone marrow mononuclear cells, it is worth noting that they can modulate immune responses through a variety of modalities. For instance, enhanced bone marrow mononuclear cells/aspirate can have direct effects on a graft or host. Such direct effects are primarily a matter of direct contact between bone marrow mononuclear cells/aspirate of the host or graft. The contact may be with structural members of the cells or with constituents in their immediate environment. Such direct mechanisms may involve direct contact, diffusion, uptake, or other processes well known to those skilled in the art. The direct activities and effects of the bone marrow mononuclear may be limited spatially, such as to an area of local deposition or to a bodily compartment accessed by injection. The invention teaches administration of augmenting agents, such as, for example, n-acetyl cysteine, leads to “enhancement” of regenerative activities of bone marrow mononuclear cells. Enhanced bone marrow mononuclear cells/aspirate also can “home” in response to “homing” signals, such as those released at sites of injury or disease. Since homing often is mediated by signals whose natural function is to recruit cells to the sites where repairs are needed, the homing behavior can be a powerful tool for concentrating enhanced bone marrow mononuclear cells/aspirate to therapeutic targets. This effect can be stimulated by specific factors, said factors including PDGF, PDAF, VEGF, PDEGF, PF-4, TGF-B, FGF-A, FGF-B, TGF-A, IGF-1, IGF-2, BTG, TSP, vWF, PAI-1, IgG, IgM, IgA, KGF, EGF, FGF, TNF, IL-1, KGF-2, fibropeptide A, fibrinogen, albumin, osteonectin, gro-alpha, vitronectin, fibrin D-dimer, favtor V, antithrombin III, a2 macroglobulin, angiogenim, Fg-D, and elastase. In further detail, growth factors, cytokines, or the like that can be present and include, without limitation, LIF, anticancer growth factors such as IGFBP3, eicosanoids such as PGs orleukotrienes, IL-1 TNF alpha, INFs, TNF-a, IL-6, IL-1(a/b), prostanoid metabolites, complement components, reactive oxygen intermediates, arachidonic acid metabolites, coagulation factors, nitrates, and chemokines. Human derived growth factors, chemokines, cytokines, and hormones can include alpha defensin, alpha synuclein, beta synuclean, 4-1BBL, 6Ckine, acidic FGF, activin A, avtivin Rib, angiopoietin 2, B-DNF, BAFF, BCA-1, BCA-1, BD-1, BMP-2, BMP-4, BMP-7, BMPRA1, BDNF, CNTF, CTGF, CTLA-4Fc, CXCL1, CXCL2, cardiotrophin-1, Cripto, Cystatin C, Dkk-1, EGF AOF, EGF, EMAP II, ENA-78, EPO, Eotaxin, FGF basic AOF, FGF-10, FGF-16, FGF 17, FGF 18, FGF19, FGF4, FGF6, FGF7, FGF8, FGF8b, FGF9, Flt3, G-CSF, GDNF, GMCSF, HGF, HGH, IFN alpha A, IFN alpha ND, IFN alpha D, IFN alpha a2b, IFN, beta 1A, IFN-gamma, IGF1, IGFII, IGFBP-4, IGFBP6, IL1alpha, IL-1Beta, IL10, IL11, IL12, IL13, IL15, IL17, IL17A. IL17F, IL18, IL19, IL2, IL20, IL21, IL23, IL28A, IL28B, IL29, IL3, IL31, IL33, IL4, ILS, IL6, IL7, IL8, IL9, IL10, ITAC, KGF2, Kallikrein11, Kallikrein4, Kallikrein7, LEFTY-A, LIF, Leptin, MCSF AOF, MCSF, MCP-1, MCP2, MCPS, MCP4, MDC, MIG, MIP1alpha, MIP1 beta, MIP3 alpha, MIP3 beta, MIP4, MIPS, midkine, NAP2, NT3, NT4, Neurotactin, neurturin, Oncostatin, osteoprotegrerin, PDGF-AA, PDGF-AB, PDGF-BB, PTN, Rank ligand, Rank receptor, RANTES<SCF, SCFAOF, SDF-1 alpha, SDF-1Beta, CD4, CD40L, TNF-RI, TNFRII, TARC, TECK, TGF alpha, TGF1 Beta1, TGF Beta2, TGF Beta3, TNF beta/lymphotoxin, TNF-alpha, TPO, TRAIL, TWEAK, and VEGF.

Enhanced bone marrow mononuclear cells/aspirate may also modulate immune processes by their response to factors. This may occur additionally or alternatively to direct modulation. Such factors may include homing factors, mitogens, and other stimulatory factors. They may also include differentiation factors, and factors that trigger particular cellular processes. Among the latter are factors that cause the secretion by cells of other specific factors, such as those that are involved in recruiting cells, such as stem cells (including Enhanced bone marrow mononuclear cells), to a site of injury or disease.

Enhanced bone marrow mononuclear cells/aspirate may, in addition to the foregoing or alternatively thereto, secrete factors that act on endogenous cells, such as stem cells or progenitor cells. The factors may act on other cells to engender, enhance, decrease, or suppress their activities. Enhanced bone marrow mononuclear cells/aspirate may secrete factors that act on stem, progenitor, or differentiated cells causing those cells to divide and/or differentiate. One such factor are exosomes and macrovesicles produced by said enhanced bone marrow mononuclear cells. Enhanced bone marrow mononuclear cells/aspirate that home to a site where repair is needed may secrete trophic factors that attract other cells to the site. In this way, enhanced bone marrow mononuclear cells/aspirate may attract stem, progenitor, or differentiated cells to a site where they are needed. Enhanced bone marrow mononuclear cells/aspirate also may secrete factors that cause such cells to divide or differentiate. Secretion of such factors, including trophic factors, can contribute to the efficacy of enhanced bone marrow mononuclear cells/aspirate in, for instance, limiting inflammatory damage, limiting vascular permeability, improving cell survival, and engendering and/or augmenting homing of repair cells to sites of damage. Such factors also may affect T-cell proliferation directly. Such factors also may affect dendritic cells, by decreasing their phagocytic and antigen presenting activities, which also may affect T-cell activity. Furthermore, such factors, or enhanced bone marrow mononuclear cells/aspirate themselves, may be capable of modulating T regulatory cell numbers.

By these and other mechanisms, enhanced bone marrow mononuclear cells/aspirate can provide beneficial immunomodulatory effects, including, but not limited to, suppression of undesirable and/or deleterious immune reactions, responses, functions, diseases, and the like. Enhanced MSC in various embodiments of the invention provide beneficial immunomodulatory properties and effects that are useful by themselves or in adjunctive therapy for precluding, preventing, lessening, decreasing, ameliorating, mitigating, treating, eliminating and/or curing deleterious immune processes and/or conditions. Such processes and conditions include, for instance, autoimmune diseases, anemias, neoplasms, HVG, GVHD, and certain inflammatory disorders. In one particular embodiment, said enhanced MSC are useful for treatment of neurological disease, inflammatory conditions, psychiatric disorders, inborn errors of metabolisms, vascular disease, cardiac disease, renal disease, hepatic disease, pulmonary disease, ocular conditions such as uveitis, gastrointestinal disorders, orthopedic disorders, dermal disorders, neoplasias, prevention of neoplasias, hematopoietic disorders, reproductive disorders, gynecological disorders, urological disorders, immunological disorders, olfactory disorders, and auricular disorders.

In one embodiment of the invention, treatment of peripheral artery disease and other conditions affecting peripheral and/or small blood vessels can also benefit from angiogenesis. All blood vessels that are surrounded by smooth muscle cells that can dilate in response to changes in nitric oxide. However, in general, the large blood vessels respond strongly to nitric oxide as compared to smaller ones. As one moves into arterioles, the vessels are more closely linked with tissue beds, these vessels are influenced to dilate not only in response to increased nitric oxide production by endothelial cells, but is also in response to regional changes in the levels of other vasodilators, compounds such as adenosine and prostaglandin 12 (these compounds act directly on smooth muscle cells to induce the nitric oxide-independent relaxation). Moreover, when endothelium is damaged or so compromised that nitric oxide is not enough to sufficiently relax the vascular system, other vasodilating agents need to be used. In one embodiment, the invention teaches the use of autologous bone marrow mononuclear cells/aspirate that are injected with augmenting agents in order to increase nitric oxide producing ability of endothelium. In another aspect of the invention, bone marrow mononuclear cells/aspirate are administered together with augmenting agents such as n-acetylcysteine, or G-CSF, in order to enhance sensitivity to nitric oxide production. For the purpose of the invention, bone marrow mononuclear cells/aspirate may be used either freshly isolated, purified, or subsequent to ex vivo culture. A typical bone marrow harvest for collecting starting material for practicing one embodiment of the invention involves a bone marrow harvest with the goal of acquiring approximately 5-700 ml of bone marrow aspirate. Numerous techniques for the aspiration of marrow are described in the art and part of standard medical practice. One particular methodology that may be attractive due to decreased invasiveness is the “mini-bone marrow harvest”. In one specific embodiment, bone marrow mononuclear cells/aspirate are isolated by pheresis or gradient centrifugation. Numerous methods of separating mononuclear cells from bone marrow are known in the art and include density gradients such as Ficoll Histopaque at a density of approximately 1.077 g/ml or Percoll gradient. Separation of cells by density gradients is usually performed by centrifugation at approximately 450 g for approximately 25-60 minutes. Cells may subsequently be washed to remove debris and unwanted materials. Said washing step may be performed in phosphate buffered saline at physiological pH. An alternative method for purification of mononuclear cells involves the use of apheresis apparatus such as the CS3000-Plus blood-cell separator (Baxter, Deerfield, USA), the Haemonetics separator (Braintree, Mass.), or the Fresenius AS 104 and the Fresenius AS TEC 104 (Fresenius, Bad Homburg, Germany) separators. In addition to injection of mononuclear cells, purified bone marrow subpopulations may be used. Additionally, ex vivo expansion and/or selection may also be utilized for augmentation of desired biological properties for use in treatment of ischemic conditions, wherein said cells are administered together with antioxidants such as n-acetylcystein.

In the methods of the present invention, autologous bone-marrow is isolated from the subject usually under general anesthesia by aspiration from the tibia, femur, ilium or sternum with a syringe, preferably containing 1 mL heparin with an 18-gauge needle. Bone-marrow mononuclear cells/aspirate are isolated using standard techniques with which one of skill is familiar; such techniques may be modified depending upon the species of the subject from which the cells are isolated. The marrow cells are transferred to a sterile tube and mixed with an appropriate amount of medium, e.g., 10 mL culture medium (Iscove's modified Dulbecco medium IMDM with 10% fetal bovine serum, penicillin G [100 U/mL] and streptomycin [100 .mu.g/mL]). The tube is centrifuged to pellet the bone marrow cells, e.g., at 2000 rpm for five minutes and the cell pellet resuspended in medium, e.g., 5 mL culture medium. Low density bone-marrow mononuclear cells/aspirate are separated from the suspension, e.g., by density gradient centrifugation over Histopaque-1083™ (Sigma), e.g. as described by Yablonka-Reuveni and Nameroff and hereby incorporated by reference. (Histochemistry (19877) 87:27-38). Briefly, the cell suspension is loaded on 20% to 60% gradient, e.g. Histopaque-1083™ (Sigma), Ficoll-Hypaque or Percoll (both available from Pharmacia, Uppsala, Sweden) according to manufacturer's instructions and as described by Yablonka-Reuveni and Nameroff. For example, the cells are centrifuged at 400 g for 20 minutes for Ficoll-Hypaque or at 2000 rpm for 10 minutes for Percoll. Following centrifugation, the top two-thirds of total volume are transferred into a tube, as these layers contain most of the low density bone-marrow mononuclear cells. The cells are centrifuged, e.g. at 2000 rpm for 10 minutes to remove the Histopaque. This is repeated and the cell pellet of bone-marrow mononuclear cells/aspirate is resuspended in culture medium or buffer, e.g., IMDM, saline, phosphate buffered saline, for transplantation. Preferably, fresh bone-marrow mononuclear cell, isolated as described above, are used for transplantation.

This invention provides a method of treating diseased tissue in a subject which comprises: a) isolating autologous bone-marrow mononuclear cells/aspirate from the subject; and b) transplanting locally into the diseased tissue an effective amount of the autologous bone-marrow mononuclear cells, thereby treating the diseased tissue in the subject. In a preferred embodiment, the diseased tissue is ischemic tissue or tissue in need of repair or regeneration. The invention teaches that augmentation of levels of antioxidants such as n-acetylcysteine, locally, or systemically in a patient receiving bone marrow mononuclear cell/aspirate administration results in increasing angiogenesis in diseased tissue in a subject. The administration of n-acetylcysteine is provided to a patient treated with a procedure which comprises: a) isolating autologous bone-marrow mononuclear cells/aspirate from the subject; and b) transplanting locally into the diseased tissue an effective amount of the autologous bone-marrow mononuclear cells, thereby increasing angiogenesis and repair in the diseased tissue in the subject. In a preferred embodiment, the tissue is ischemic tissue or tissue in need of repair or regeneration. This invention also provides a method of preventing heart failure in a subject which is treated with n-acetylcysteine and further subjected to a procedure comprising: a) isolating autologous bone-marrow mononuclear cells/aspirate from the subject; and b) transplanting locally into heart tissue an effective amount of the autologous bone-marrow mononuclear cells/aspirate so as to result in formation of new blood vessels in the heart tissue, to increase angiogenesis and repair in the heart tissue in the subject, thereby preventing heart failure in the subject. In a preferred embodiment, the heart tissue is ischemic heart tissue or heart tissue in need of repair or regeneration after injury or surgery. In other preferred embodiments, compromised or occluded coronary blood vessels are treated by the above-described methods resulting in formation of new blood vessels.

The invention provides a method of utilizing n-acetylcysteine administration, either locally, systemically, or in delayed release form for the purpose of augmentation of tissue regeneration in a subject which comprises: a) isolating autologous bone-marrow mononuclear cells/aspirate from the subject; and b) transplanting locally into the tissue an effective amount of the autologous bone-marrow mononuclear cells, resulting in formation of new blood vessels in the tissue, i.e. increasing angiogenesis and repair in diseased tissue in the subject. In a preferred embodiment, the tissue is diseased tissue. More preferably, the diseased tissue is ischemic tissue or damaged tissue in need of repair or regeneration.

In some embodiments, the bone-marrow mononuclear cells/aspirate may also be cultured in any complete medium containing up to 10% serum, e.g., IMDM containing 10% fetal bovine serum and antibiotics, as described above, for up to four weeks before transplantation. The cells may be cultured with growth factors, e.g., vascular endothelial growth factor. The medium is changed about twice a week. The cultured cells are dissociated from the culture dishes with 0.05% trypsin (Gibco BRL, Grand Island, N.Y.), neutralized with culture medium and collected by centrifugation, for example, at 2000 rpm for five minutes at room temperature. The cells are resuspended in IMDM at a concentration of .apprxeq.1.times.10.sup.5 cells to about 1.times.10.sup.10 cells, preferably about 1.times.10.sup.7 cells to about 1.times.10.sup.8 cells in 50 .mu.L for transplantation.

In some embodiments of the invention, it is important to assess the efficacy of augmented responsiveness to vasodilatory agents as a means of assessing endothelial function. It is possible to measure endothelial function by measuring vasodilatation after intra-arterial pharmacologic stimulation with substances that enhance the release of endothelial nitric oxide (such as acetylcholine and bradykinin). Therefore, noninvasive tests of endothelial function have come into existence. One based on ultrasound measures flow-mediated changes in arterial diameter in relatively superficial arteries, such as the brachial, radial or femoral vessels. Thus, this technique measures endothelial function in conduit arteries rather than resistance vessels. Flow-mediated changes in conduit artery diameter are caused by shear-stress induced generation of endothelial derived vasoactive mediators (flow-mediated dilatation). Since the arterial dilator response to shear-stress can be almost completely blocked by pretreatment with nitric oxide synthase inhibitors [1], it has been suggested that the phenomenon is predominantly due to endothelial release of nitric oxide. However, endothelial function assessed by this method correlates significantly with invasive testing of coronary endothelial function [2], as well as with the severity and extent of coronary atherosclerosis [3, 4]. Accordingly, in one embodiment of the invention, noninvasive endothelial function testing has provided valuable insights into vascular changes associated with early atherogenesis and the potential reversibility of arterial disease.

The invention teaches the use of bone marrow mononuclear cells/aspirate administered in various forms for stimulation of regeneration in patients in need of treatment. Examples of patients (e.g. a human or a veterinary animal) in need of stimulation of regeneration of peripheral blood vessels are those suffering from, or at risk of suffering from, diminished blood flow in such blood vessels. For example, a subject may suffer and/or be at risk of, peripheral vascular disease, e.g. Raynaud's disease, peripheral artery disease (PAD), intermittent claudication (found in subjects suffering from early stages of PAD, this condition results from decreased blood flow to the legs during periods of exercise, including walking/moving around, and causing pain, fatigue or other discomfort in the affected muscle; the discomfort dissipates with the cessation of the activity), vasculitis of small blood vessels, vasospasm, venous thrombosis, venous insufficiency, lymphatic disorders (e.g. lymphatic insufficiency), critical limb ischemia (severe obstruction of the arteries which decreases blood flow to the hands, feet, and legs; one of the symptoms of PAD), acute limb ischemia (an arterial occlusion which suddenly limits blood flow to the arm or leg), atheroembolism (an embolism of lipid debris from an ulcerated atheromatous deposit), and/or lower extremity ischemia (an occlusive disease in arteries supplying blood to lower extremities causing inadequate blood flow).

In one embodiment, the invention teaches the use of bone marrow mononuclear cells/aspirate administered together with n-acetylcysteine as a method of treating or preventing a condition or conditions selected from the group consisting of Raynaud's disease, peripheral artery disease (PAD), intermittent claudication, vasculitis of small blood vessels, vasospasm, venous thrombosis, venous insufficiency, lymphatic disorders (e.g. lymphatic insufficiency), critical limb ischemia, acute limb ischemia, atheroembolism, and lower extremity ischemia.

In one embodiment, the invention teaches the treatment of ischemic disease using administration of n-acetylcysteine together with autologous bone marrow mononuclear cells, administration of this combination is used to treat the ischemia, said ischemia is selected from the group consisting of myocardial ischemia, cerebral ischemia, renal ischemia, liver ischemia, peripheral muscle tissue ischemia, retinal ischemia and spinal cord ischemia. As described supra, ischemia may occur in any tissue and/or organ suffering from a lack of oxygen and/or metabolites for a prolonged time which results in organic defects. The term “organ defect” as used herein relates to dysfunctional myocardium, brain, kidney, liver, peripheral muscle, retina or spinal cord defects. Said organ defects are caused by myocardial ischemia, e.g., due to heart failure, hypertension, coronary artery disease (CAD), myocardial infarction, thrombo-embolic events, trauma and/or surgical procedures; cerebral ischemia, e.g., due to trauma, stroke, thrombo-embolic events, malformation of blood-supplying vessels, multi-infarct disease, cerebral hemorrhage, surgical and/or interventional measures; renal ischemia, e.g., due to thrombo-embolic events, atherosclerosis, malformation of blood-supplying vessels, trauma and/or surgical procedures; liver ischemia, e.g., due thrombo-embolic events, malformation of blood-supplying vessels, trauma and/or surgical procedures; peripheral muscle tissue ischemia, e.g., is caused by thrombo-embolic events, atherosclerosis, malformation of blood-supplying vessels, trauma and/or surgical procedures; retinal ischemia, e.g., is caused by thrombo-embolic events, malformation of blood-supplying vessels, trauma and/or surgical procedures; and spinal cord ischemia, e.g., is caused by thrombo-embolic events, atherosclerosis, malformation of blood-supplying vessels, trauma and/or surgical procedures. The myocardial ischemia which is treated with the present invention is caused by heart failure, hypertension, coronary artery disease (CAD), myocardial infarction, thrombo-embolic events, trauma and/or surgical procedures. The cerebral ischemia which is treated with the present invention is caused by trauma, stroke, thrombo-embolic events, malformation of blood-supplying vessels, multi-infarct disease, cerebral hemorrhage, surgical and/or interventional measures. The renal ischemia which is treated with the present invention is caused by thrombo-embolic events, atherosclerosis, malformation of blood-supplying vessels, trauma and/or surgical procedures. The liver ischemia or retinal ischemia which is treated with the present invention is caused by thrombo-embolic events, malformation of blood-supplying vessels, trauma and/or surgical procedures. The peripheral muscle tissue ischemia or spinal cord ischemia which is treated with the present invention is caused by thrombo-embolic events, atherosclerosis, malformation of blood-supplying vessels, trauma and/or surgical procedures.

In a one embodiment, the present invention provides method of treating a subject having an ischemic tissue or a tissue damaged by ischemia comprising of: administering a therapeutically effective amount of a composition comprising bone marrow mononuclear cells/aspirate co-injected with n-acetylcysteine, wherein said n-acetylcysteine is administered by time release means. Furthermore, in one embodiment, said bone marrow mononuclear cells/aspirate may be treated ex vivo with a prostaglandin pathway agonist and optionally, a glucocorticoid, under conditions sufficient to increase CXCR4 gene expression at least two-fold in the treated stem or progenitor cells compared to non-treated bone marrow mononuclear cells. In a certain embodiment, the present invention contemplates, in part, a method of ameliorating at least one symptom associated with an ischemic tissue or a tissue damaged by ischemia in a subject comprising of: administering a therapeutically effective amount of a composition comprising of bone marrow mononuclear cells/aspirate treated ex vivo with a prostaglandin pathway agonist and optionally, a glucocorticoid, under conditions sufficient to increase CXCR4 gene expression at least two fold in the treated stem or progenitor cells compared to non-treated bone marrow mononuclear cells, wherein said cells are administered together with n-acetylcysteine. In some embodiments, n-acetylcysteine is utilized as a means of increasing expression of CXCR4. Furthermore, in one embodiment, the present invention contemplates, in part, a method of increasing bone marrow mononuclear cell homing to an ischemic tissue or a tissue damaged by ischemia. This comprises of treating stem or progenitor cells ex vivo with a prostaglandin pathway agonist and optionally, a glucocorticoid, under conditions sufficient to increase the percent (%) migration in an SDF-1 transwell migration assay at least two fold in the treated stem or progenitor cells compared to non-treated stem or progenitor cells; and administering a composition comprising said bone marrow mononuclear cells/aspirate to a subject having an ischemic tissue or a tissue damaged by ischemia. In some embodiments of the invention, n-acetylcysteine is administered ex vivo to enhance migration towards SDF-1 alone, or through coadministration with said prostaglandin pathway inhibitor and/or glucocorticoids.

In one embodiment of the invention, bone marrow mononuclear cells/aspirate are genetically modified to enhance desirable properties of the cells. For modification, in some embodiments, specific types of cells with regenerative activity may be isolated from said bone marrow mononuclear cells/aspirate before transfection. In one embodiment of the invention cells are transfected with anti-apoptotic proteins to enhance in vivo longevity. The present invention includes a method of using cells that have been cultured under conditions to express increased amounts of at least one anti-apoptotic protein as a therapy to inhibit or prevent apoptosis. In one embodiment, the cells which are used as a therapy to inhibit or prevent apoptosis have been contacted with an apoptotic cell. The invention is based on the discovery that bone marrow that has been contacted with n-acetylcysteine expresses high levels of anti-apoptotic molecules. In some instances, the bone marrow cells that have been contacted with an apoptotic cell secrete high levels of at least one anti-apoptotic protein, including but not limited to, STC-1, BCL-2, XIAP, Survivin, and Bcl-2XL. Methods of transfecting antiapoptotic genes into cells have been previously described which can be applied to the current invention. Said antiapoptotic genes that can be utilized for practice of the invention, in a nonlimiting way, include GATA-4 [5], FGF-2 [6], bcl-2 [7, 8], and HO-1 [9].

The autologous bone-marrow mononuclear cells/aspirate are transplanted by injection into the center, bordering zone, or neighboring areas of the ischemic tissue. In additional embodiments of the present invention, the autologous bone-marrow mononuclear cells/aspirate may be transplanted into or near any site of any tissue in which angiogenesis or repair is required. Such tissue includes but is not limited to under perfused tissue of any end-organ, e.g. tissues with chronic ischemia. Such under perfused tissue includes but is not limited to the heart, brain, skeletal muscle, kidney, liver, organs of the gastrointestinal tract and other organs and tissues requiring repair. The transplanted autologous bone-marrow mononuclear cells/aspirate are delivered to the desired tissue site(s) in an effective amount of .apprxeq.1.times.10.sup.5 cells to about 1.times.10.sup.10 cells, preferably about 1.times.10.sup.7 cells to about 1.times.10.sup.8 cells, per injection site, preferably by needle injection. Preferably, a tissue receives a total of about fifty injections, e.g. for a leg or arm, and about ten injections into heart muscle. Alternatively, the autologous bone-marrow mononuclear cells/aspirate are delivered by intravascular injection or infusion into arteries or veins, endoluminal injection directly into an occlusion, retrograde perfusion, pericardial delivery, implants (biodegradable or biostable), e.g. local implant scaffold, patch, needle-free injection using propulsion by gas such as CO.sub.2, acceleration or transfer into tissue by other means such as iontophoresis or electroporation, pressure or application to a tissue or organ surface. In general, delivery may be accomplished with the use of any medical device for delivery of transplanted cells. In preferred embodiments of any of the methods described herein, the tissue into which autologous bone-marrow mononuclear cells/aspirate are transplanted includes any diseased or damaged tissue and any tissue in need of repair or regeneration, including but not limited to under perfused tissue such as tissue found in chronic ischemia. Preferably, the tissue includes but is not limited to ischemic tissue. More preferably the tissue includes such tissue as cardiac muscle tissue, skeletal muscle tissue, brain tissue e.g., affected by stroke or AV malformations, coronary vessels, kidney, liver, organs of the gastrointestinal tract, muscle tissue afflicted by atrophy, including neurologically based muscle atrophy. In further embodiments the subject is preferably a mammal. Most preferably, the mammal is a human.

In the present invention, autologous bone marrow mononuclear cells/aspirate is locally transplanted into ischemic tissues. There are several advantages of local transplantation rather than intravenous transfusion of bone marrow mononuclear cells/aspirate for therapeutic neovascularization. First, through local transplantation, one can increase the density of endothelial progenitor cells at the target tissue compared with intravenous infusion. In the present invention, .apprxeq.1.times.10.sup.5 cells to about 1.times.10.sup.10 cells, preferably about 1.times.10.sup.7 cells to about 1.times.10.sup.8 cells per injection site are delivered, preferably by needle injection within or near the diseased or damaged tissue or any tissue in need of repair or tissue regeneration, e.g. ischemic tissues. This may be an advantage for cell survival in the tissues, because it is believed that cells must form clusters to survive in tissues. The local transplantation may reduce the systemic side effects of transplanted bone marrow mononuclear cells/aspirate compared with systemic infusion. Other preferred means of delivery of autologous bone marrow mononuclear cells/aspirate to the tissue include but are not limited to delivery by intravascular injection or infusion into arteries or veins, endoluminal injection directly into an occlusion, retrograde perfusion, pericardial delivery, implants (biodegradable or biostable), e.g. local implant scaffold, patch, needle-free injection using propulsion by gas such as CO.sub.2, acceleration or transfer into tissue by other means such as iontophoresis or electroporation, pressure or application to a tissue or organ surface. In general, delivery may be accomplished with the use of any medical device for delivery of transplanted cells. Preferably each tissue receives a total of about ten to fifty injections.

For the practice of the invention autologous bone marrow mononuclear cells/aspirate are transplanted to an ischemic tissue where they become incorporated into or participate in the formation of new blood vessels and/or capillaries. Alternatively, said bone marrow mononuclear cells/aspirate may provide trophic support for augmentation of activity of residing progenitor cells.

The choice of formulation for administering bone marrow derived mononuclear cells/aspirate for a given application will depend on a variety of factors. Prominent among these will be the species of subject, the nature of the disorder, dysfunction, or disease being treated and its state and distribution in the subject, the nature of other therapies and agents that are being administered, the optimum route for administration of the bone marrow derived mononuclear cells, survivability of bone marrow derived mononuclear cells/aspirate via the route, the dosing regimen, and other factors that will be apparent to those skilled in the art. In particular, for instance, the choice of suitable carriers and other additives will depend on the exact route of administration and the nature of the particular dosage form, for example, liquid dosage form (e.g., whether the composition is to be formulated into a solution, a suspension, gel or another liquid form, such as a time release form or liquid-filled form).

For example, cell survival can be an important determinant of the efficacy of cell-based therapies. This is true for both primary and adjunctive therapies. Another concern arises when target sites are inhospitable to cell seeding and cell growth. This may impede access to the site and/or engraftment there of therapeutic bone marrow derived mononuclear cells/aspirate. Various embodiments of the invention comprise measures to increase cell survival and/or to overcome problems posed by barriers to seeding and/or growth.

Examples of compositions comprising bone marrow derived mononuclear cells include liquid preparations, including suspensions and preparations for intramuscular or intravenous administration (e.g., injectable administration), such as sterile suspensions or emulsions. Such compositions may comprise an admixture of bone marrow derived mononuclear cells with a suitable carrier, diluent, or excipient such as sterile water, physiological saline, glucose, dextrose, or the like. The compositions can also be lyophilized. The compositions can contain auxiliary substances such as wetting or emulsifying agents, pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE,” 17th edition, 1985, incorporated herein by reference, may be consulted to prepare suitable preparations, without undue experimentation.

Compositions of the invention often are conveniently provided as liquid preparations, e.g., isotonic aqueous solutions, suspensions, emulsions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Various additives often will be included to enhance the stability, sterility, and isotonicity of the compositions, such as antimicrobial preservatives, antioxidants, chelating agents, and buffers, among others. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents that delay absorption, for example, aluminum monostearate, and gelatin. According to the present invention, however, any vehicle, diluent, or additive used would have to be compatible with the cells. Bone marrow derived mononuclear cells solutions, suspensions, and gels normally contain a major amount of water (preferably purified, sterilized water) in addition to the cells. Minor amounts of other ingredients such as pH adjusters (e.g., a base such as NaOH), emulsifiers or dispersing agents, buffering agents, preservatives, wetting agents and jelling agents (e.g., methylcellulose) may also be present. Typically, the compositions will be isotonic, i.e., they will have the same osmotic pressure as blood and lacrimal fluid when properly prepared for administration. The desired isotonicity of the compositions of this invention may be accomplished using sodium chloride, or other pharmaceutically acceptable agents such as dextrose, boric acid, sodium tartrate, propylene glycol, or other inorganic or organic solutes. Sodium chloride is preferred particularly for buffers containing sodium ions. Viscosity of the compositions, if desired, can be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount, which will achieve the selected viscosity. Viscous compositions are normally prepared from solutions by the addition of such thickening agents.

A pharmaceutically acceptable preservative or cell stabilizer can be employed to increase the life of bone marrow derived mononuclear cells compositions. If such preservatives are included, it is well within the purview of the skilled artisan to select compositions that will not affect the viability or efficacy of the bone marrow derived mononuclear cells.

Those skilled in the art will recognize that the components of the compositions should be chemically inert. This will present no problem to those skilled in chemical and pharmaceutical principles. Problems can be readily avoided by reference to standard texts or by simple experiments (not involving undue experimentation) using information provided by the disclosure, the documents cited herein, and generally available in the art.

Sterile injectable solutions can be prepared by incorporating the cells utilized in practicing the present invention in the required amount of the appropriate solvent with various amounts of the other ingredients, as desired.

In some embodiments, bone marrow derived mononuclear cells/aspirate are formulated in a unit dosage injectable form, such as a solution, suspension, or emulsion. Pharmaceutical formulations suitable for injection of bone marrow derived mononuclear cells typically are sterile aqueous solutions and dispersions. Carriers for injectable formulations can be a solvent or dispersing medium containing, for example, water, saline, phosphate buffered saline, polyol (for example, glycerol, propylene glycol, liquid polyethylene glycol, and the like), and suitable mixtures thereof.

The skilled artisan can readily determine the number of cells and optional additives, vehicles, and/or carrier in compositions to be administered in methods of the invention. Typically, any additives (in addition to the cells) are present in an amount of 0.001 to 50 wt % in solution, such as in phosphate buffered saline. The active ingredient is present in the order of micrograms to milligrams, such as about 0.0001 to about 5 wt %, preferably about 0.0001 to about 1 wt %, most preferably about 0.0001 to about 0.05 wt % or about 0.001 to about 20 wt %, preferably about 0.01 to about 10 wt %, and most preferably about 0.05 to about 5 wt %. The cells may also be given as a direct undiluted aspirate which is not filtered.

For any composition to be administered to an animal or human, and for any particular method of administration, it is preferred to determine therefore: toxicity, such as by determining the lethal dose (LD) and LD50 in a suitable animal model, e.g., rodent such as mouse or rat; and, the dosage of the composition(s), concentration of components therein, and timing of administering the composition(s), which elicit a suitable response. Such determinations do not require undue experimentation from the knowledge of the skilled artisan, this disclosure, and the documents cited herein. And, the time for sequential administrations can be ascertained without undue experimentation.

In some embodiments, bone marrow derived mononuclear cells/aspirate are encapsulated for administration, particularly where encapsulation enhances the effectiveness of the therapy, or provides advantages in handling and/or shelf life. Encapsulation in some embodiments where it increases the efficacy of bone marrow derived mononuclear cells mediated immunosuppression may, as a result, also reduce the need for immunosuppressive drug therapy.

Also, encapsulation in some embodiments provides a barrier to a subject's immune system that may further reduce a subject's immune response to the bone marrow derived mononuclear cells (which generally are not immunogenic or are only weakly immunogenic in allogeneic transplants), thereby reducing any graft rejection or inflammation that might occur upon administration of the cells.

In a variety of embodiments where bone marrow derived mononuclear cells/aspirate are administered in admixture with cells of another type, which are more typically immunogenic in an allogeneic or xenogeneic setting, encapsulation may reduce or eliminate adverse host immune responses to the non-enhanced MSC cells and/or GVHD that might occur in an immunocompromised host if the admixed cells are immunocompetent and recognize the host as non-self. In some embodiments, bone marrow derived mononuclear cells may be encapsulated by membranes, as well as capsules, prior to implantation. It is contemplated that any of the many methods of cell encapsulation available may be employed. In some embodiments, cells are individually encapsulated. In some embodiments, many cells are encapsulated within the same membrane. In embodiments in which the cells are to be removed following implantation, a relatively large size structure encapsulating many cells, such as within a single membrane, may provide a convenient means for retrieval. A wide variety of materials may be used in various embodiments for microencapsulation of Enhanced bone marrow derived mononuclear cells. Such materials include, for example, polymer capsules, alginate-poly-L-lysine-alginate microcapsules, barium poly-L-lysine alginate capsules, barium alginate capsules, polyacrylonitrile/polyvinylchloride (PAN/PVC) hollow fibers, and polyethersulfone (PES) hollow fibers. Techniques for microencapsulation of cells that may be used for administration of enhanced MSC are known to those of skill in the art and are described, for example, in U.S. Pat. No. 5,639,275 (which, for example, describes a biocompatible capsule for long-term maintenance of cells that stably express biologically active molecules). Additional methods of encapsulation are in European Patent Publication No. 301,777 and U.S. Pat. Nos. 4,353,888; 4,744,933; 4,749,620; 4,814,274; 5,084,350; 5,089,272; 5,578,442; 5,639,275; and 5,676,943. All of the foregoing are incorporated herein by reference in parts pertinent to encapsulation of bone marrow derived mononuclear cells.

Certain embodiments incorporate bone marrow derived mononuclear cells/aspirate into a polymer, such as a biopolymer or synthetic polymer. Examples of biopolymers include, but are not limited to, fibronectin, fibin, fibrinogen, thrombin, collagen, and proteoglycans. Other factors, such as the cytokines discussed above, can also be incorporated into the polymer. In other embodiments of the invention, bone marrow derived mononuclear cells may be incorporated in the interstices of a three-dimensional gel. A large polymer or gel, typically, will be surgically implanted. A polymer or gel that can be formulated in small enough particles or fibers can be administered by other common, more convenient, non-surgical routes.

In one embodiment, reduction of inflammation to enhance stem cell efficacy in the ischemic tissue is achieved by administration of various agents which include redox-active compounds, iron-ligand inhibitors, flavonoids, corticosteroids, and nonsteroidal anti-inflammatory drugs. Compounds or molecules that reduce reactive oxygen species, oxidation of lipids, and lipid peroxidation which include various antioxidants which could be used in combination are also preferably added to the solution. Examples include zinc, vitamin C (ascorbic acid), vitamin E (alpha-tocopherol), selenium, Trolox, ebselen, glutathione, carotenes, ubiquinol (Coenzyme Q), propyl gallate (PG), butylated hydroxytoluene (BHT), butylated hydroxyanisole (BHA), hydrogen sulfide, erythrobate, sodium tripolyphosphate, ethylenediaminetetraacetic acid, ethoxyquin, caseinates, pyruvate, natural herbs, honey, and similar compounds that inhibit oxidation. Other immune modulators that may be used together with antioxidants to modify the articular niche include: FAS ligand, IL-2R, IL-1 Ra, IL-2, IL-4, IL-8, IL-10, IL-20, IL-35, HLA-G, PD-L1, 1-309, IDO, iNOS, CD200, Galectin 3, sCR1, arginase, PGE-2, aspirin, atorvastatin, fluvastatin, lovastatin, pravastatin, rosuvastatin, simvastatin, pitavastatin, n-acetylcysteine, rapamycin, IVIG, naltrexone, TGF-beta, VEGF, PDGF, CTLA-4, anti-CD45RB antibody, hydroxychloroquine, leflunomide, auranofin, dicyanogold, sulfasalazine, methotrexate, glucocorticoids, etanercept, adalimumab, abatacept, anakinra, certolizumab, Etanercept-szzs, golimumab, infliximab, rituximab, tocilizumab, cyclosporine, IFN-gamma, everolimus, rapamycin, or combinations thereof.

In other embodiments, inhibitors of nitric oxide are utilized to modulate the microenvironment of the ischemic tissue before, during and after stem cell administration. Comprised nitric oxide synthase inhibitors and nitric oxide scavengers comprising; arginine derivatives, methylated arginines, substituted L-arginine, nitro-arginine, L-N.sup.G-nitroarginine, N.sup.G-mono-methyl-L-arginine (L-NMMA), N-nitro-L-arginine methyl ester (L-NAME), N-amino-L-arginine, N-methyl-L-arginine, N.sup.G-monomethyl-L-arginine (L-NMA), N.sup.G-nitro-L-arginine (L-NNA), aminoguanidine, 7-nitroindazole, S-ethylisothiourea, S-methylisothiourea, S-methylthiocitriulline, S-ethylthiocitrulline, N-ethylimino-L-ornithine, N-iminoethyl-L-lysine (L-NIL), flavoprotein binders. diphenyleneiodonium and related iodonium derivatives, omithine and omithine derivatives; tetracycline and derivaties thereof; L-canavanine; citrulline; redox dyes, methylene blue; calmodulin binders, trifluoropiperazine and calcinarin; heme binders; resveratrol; zinc compounds; tetrahydropterin analogs, aminoguanidine; and depleters of biopterin, methotrexate, N-acetylcysteine, nonsteroidal anti-inflammatory agents, sodium salicylate, and mixtures thereof.

In some embodiments, other pharmaceutically active ingredients are utilized to modulate the arthritic microenvironment including: pharmaceutically active agents selected from the group consisting of growth factors, differentiation factors, enzymes, receptor agonists or antagonists, antibodies, hormones, analgesics, local anesthetics, anti-inflammatory drugs, such as Indomethacin and tiaprofenic acid, TNF-.alpha. inhibitors, antibiotics, anti-microbial agents; antibiotics; antiproliferative, cytotoxic, and antitumor drugs including chemotherapeutic drugs; analgesic; antiangiogen; antibody; antivirals; cytokines; colony stimulating factors; proteins; chemoattractants; EDTA; histamine; antihistamine; erythropoietin; antifungals; antiparasitic agents; non-corticosteroid anti-inflammatory agents; anticoagulants; anesthetics including local anesthetics such as lidocaine and bupivicaine; analgesics; oncology agents; cardiovascular drugs; nutritional supplements; hormones; glycoproteins; fibronectin; peptides; interferons; cartilage inducing factors; protease inhibitors; vasoconstrictors, vasodilators, demineralized bone or bone morphogenetic proteins; hormones; lipids; carbohydrates; proteoglycans, versican, decorin, and biglycan; antiangiogenins; antigens; DBM; hyaluronic acid and salts and derivatives thereof; polysaccharides; cellulose compounds; antibodies; gene therapy reagents; genetically altered cells, stem cells; cell growth factors; type I and II collagen; collagen hydrolysate; elastin; sulfated glycosaminoglycan (sGAG), glucosamine sulfate; pH modifiers; methylsulfonylmethane (MSM); osteogenic compounds; osteoconductive compounds; plasminogen; nucleotides; oligonucleotides; polynucleotides; polymers; osteogenic protein 1 (OP-1 including recombinant OP-1); LMP-1 (Lim Mineralization Protein-1); cartilage including autologous cartilage; oxygen-containing components; enzymes such as, for example, peroxidase, which mediate the release of oxygen from such components; melatonin; vitamins; nutrients, and combinations thereof.

### REFERENCES

ADDIN EN.REFLIST 1. Joannides, R., et al., Nitric oxide is responsible for flow-dependent dilatation of human peripheral conduit arteries in vivo. Circulation, 1995. 91(5): p. 1314-9.
2. Takase, B., et al., Endothelium-dependent flow-mediated vasodilation in coronary and brachial arteries in suspected coronary artery disease. Am J Cardiol, 1998. 82(12): p. 1535-9, A7-8.
3. Jambrik, Z., et al., Peripheral vascular endothelial function testing for the diagnosis of coronary artery disease. Am Heart J, 2004. 148(4): p. 684-9.
4. Neunteufl, T., et al., Systemic endothelial dysfunction is related to the extent and severity of coronary artery disease. Atherosclerosis, 1997. 129(1): p. 111-8.
5. Yu, B., et al., Enhanced mesenchymal stem cell survival induced by GATA-4 overexpression is partially mediated by regulation of the miR-15 family. Int J Biochem Cell Biol, 2013. 45(12): p. 2724-35.
6. Xu, W., et al., Basic fibroblast growth factor expression is implicated in mesenchymal stem cells response to light-induced retinal injury. Cell Mol Neurobiol, 2013. 33(8): p. 1171-9.
7. Fang, Z., et al., Differentiation of GFP-Bcl-2-engineered mesenchymal stem cells towards a nucleus pulposus-like phenotype under hypoxia in vitro. Biochem Biophys Res Commun, 2013. 432(3): p. 444-50.
8. Li, W., et al., Bcl-2 engineered MSCs inhibited apoptosis and improved heart function. Stem Cells, 2007. 25(8): p. 2118-27.
9. Tsubokawa, T., et al., Impact of anti-apoptotic and anti-oxidative effects of bone marrow mesenchymal stem cells with transient overexpression of heme oxygenase-1 on myocardial ischemia. Am J Physiol Heart Circ Physiol, 2010. 298(5): p. H1320-9.

1. A method of treating an ischemic disease in a patient in need comprising administration of bone marrow mononuclear cells/aspirate in a microenvironment that has been altered in order to augment the efficacy of said bone marrow mononuclear cells/aspirate to induce a therapeutic benefit in said ischemic disease with the patient in need.

2. The method of claim 1, wherein said ischemic disease is heart failure.

3. The method of claim 1, wherein said ischemic disease is stroke.

4. The method of claim 1, wherein said ischemic disease is limb ischemia.

5. The method of claim 1, wherein said ischemic disease is buerger's disease.

6. The method of claim 1, wherein said antioxidant is super oxide dismutase.

7. The method of claim 6, wherein said super oxide dismutase is manganese dependent.

8. The method of claim 1, wherein said antioxidant is glutathione.

9. The method of claim 1, wherein said antioxidant is hydrogen gas.

10. The method of claim 1, wherein said antioxidant is xenon gas.

11. The method of claim 1, wherein said antioxidant is minocycline.

12. The method of claim 1, wherein said antioxidant is tetracycline.

13. The method of claim 1, wherein said anti-inflammatory agent is hydroxychloroquine.

14. The method of claim 1, wherein said anti-inflammatory agent is an NF-kappa B inhibitor.

15. The method of claim 14, wherein said NF-kappa B inhibitor is hydroxychloroquine.

16. The method of claim 14, wherein said NF-kappa B inhibitor is Calagualine.

17. The method of claim 14, wherein said NF-kappa B inhibitor is Conophylline.

18. The method of claim 14, wherein said NF-kappa B inhibitor is Evodiamine.

19. The method of claim 14, wherein said NF-kappa B inhibitor is Geldanamycin.

20. The method of claim 14, wherein said NF-kappa B inhibitor is selected from the group consisting of: Perrilyl alcohol, Protein-bound polysaccharide from basidiomycetes, Rocaglamides (Aglaia derivatives), 15-deoxy-prostaglandin J(2), Lead, Anandamide, Artemisia vestita, Cobrotoxin, Dehydroascorbic acid (Vitamin C), Herbimycin A, Isorhapontigenin, Manumycin A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2C1), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin A1, 20(S)-Protopanaxatriol (ginsenoside metabolite), Rengyolone, Rottlerin, and Saikosaponin-d, Saline (low Na+ isotonic).
